(19) United States
(12) Patent Application Publication
(10) Pub. No.: US 2007/0032879 A1
(43) Pub. Date: Feb. 8, 2007

(54) Anti-buckling sleeve

(76) Inventors: Andy H. Levine, Newton, MA (US); David A. Melanson, Hudson, NH (US); Ian Parker, Bristol, RI (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/494,284

(22) Filed: Jul. 27, 2006

Related U.S. Application Data
(63) Continuation-in-part of application No. 11/000,099, filed on Nov. 30, 2004.
Application No. 11/000,099 is a division of application No. 10/339,786, filed on Jan. 9, 2003, now Pat. No. 7,025,791.
(63) Continuation-in-part of application No. 11/147,984, filed on Jun. 8, 2005.
(60) Provisional application No. 60/430,321, filed on Dec. 2, 2002.
(60) Provisional application No. 60/645,296, filed on Jan. 19, 2005.
(60) Provisional application No. 60/662,570, filed on Mar. 17, 2005.

(51) Int. Cl. A61F 2/04; A61F 2/82

(52) U.S. Cl. 623/23.7; 623/1.36

(57) ABSTRACT

The invention relates to improved means for preventing buckling and therefore eversion of thin-walled, flexible, floppy gastrointestinal liners implanted in the digestive tract of an animal. The implantable devices include an anchor adapted for attachment within a natural body lumen and a thin-walled, floppy sleeve open at both ends and defining a lumen therebetween. A substantial length of the sleeve has material characteristics that result in the sleeve being prone to buckling and therefore eversion in the presence of retrograde pressures. Exemplary anti-buckling mechanisms provide an increased stiffness and/or an increased friction coefficient between the anchor and the proximal end of the sleeve to resist buckling and therefore eversion. In some embodiments, the anti-buckling mechanism is as a wire coupled along the substantial length of the sleeve.

### RELATED APPLICATIONS

This application is a continuation-in-part of U.S. application Ser. No. 11/000,099, filed Nov. 30, 2004, which is a divisional of U.S. application Ser. No. 10/339,786, filed Jan. 9, 2003, which claims the benefit of U.S. Provisional Application No. 60/430,321, filed on Dec. 2, 2002. This application is also a continuation-in-part of U.S. application Ser. No. 11/147,984, filed on Jun. 8, 2005, which claims the benefit of U.S. Provisional 60/645,296, filed on Jan. 19, 2005 and U.S. Provisional 60/662,570, filed on Mar. 17, 2005. The entire teachings of the above applications are incorporated herein by reference.

### BACKGROUND OF THE INVENTION

According to the Center for Disease Control (CDC), over sixty percent of the United States population is overweight, and almost twenty percent are obese, presenting an overwhelming health problem. Moreover, obesity-related conditions cause as many as 280,000 deaths per year, generate $51 billion in annual US healthcare costs, and cause Americans to spend $33 billion per year on weight loss products. For example, one of the principle costs to the healthcare system stems from the co-morbidities associated with obesity. Type-2 diabetes has climbed to 7.3% of the population. Of those persons with Type-2 diabetes, almost half are clinically obese, and two thirds are approaching obese. Other co-morbidities include hypertension, coronary artery disease, hypercholesteremia, sleep apnea and pulmonary hypertension.

Two surgical procedures commonly performed that successfully produce long-term weight loss are the Roux-en-Y gastric bypass and the biliopancreatic diversion with duodenal switch (BPD). Both procedures reduce the size of the stomach plus shorten the effective-length of intestine available for nutrient absorption. However, these are serious surgical procedures with significant side effects, and thus they are reserved for the most morbidly obese.

Other devices to reduce absorption in the small intestines have been proposed (See U.S. Pat. No. 5,820,584 (Crabb), U.S. Pat. No. 5,306,300 (Berry) and U.S. Pat. No. 4,315,509 (Smit)). However, these devices are yet to be successfully implemented.

### SUMMARY OF THE INVENTION

Examples of gastrointestinal sleeves have been described, which have great promise for treating obesity while minimizing the risks of surgery (See, for example, Meade et al., U.S. Utility Application Ser. No. 10/339,786, filed Jun. 9, 2003; the entire teachings of which are incorporated herein by reference). It is important in any intestinal sleeve application to maintain unobstructed pressure through the device. When a sleeve is subjected to retrograde pressure, the sleeve may tend to buckle and thus evert. Such eversions are undesirable and may lead to blockage, sleeve damage, and related complications. Thus, further improvements are desired to more fully realize the advantages which can be provided by gastrointestinal sleeves while minimizing any risk of complications.

This invention relates to improved methods and devices for preventing buckling of a thin-walled, floppy sleeve implant, anchored within a natural lumen of an animal body. The device may include an anchor adapted for attachment within a natural body lumen and a thin-walled, floppy sleeve open at both ends and defining a lumen therebetween. The device may also include an anti-buckling mechanism that extends from below a distal end of the anchor along a length of the thin walled, floppy sleeve. The length of the sleeve may extend to the distal end of the sleeve.

In one embodiment, the anti-buckling mechanism provides increased stiffness relative to the sleeve's stiffness. Some ways of increasing stiffness include providing a different material that is stiffer than the sleeve itself. Alternatively, or in addition, stiffness can be increased by providing a reinforcing member. For example, one or more soft, flexible wires can be coupled to the proximal end of the sleeve adjacent to the anchor.

The invention also relates to a method for inhibiting buckling of a sleeve when it is implanted within a natural body lumen. The method includes the steps of providing an anti-buckling mechanism that is coupled to a thin-walled, floppy sleeve. Next, a proximal end of the sleeve coupled to the anti-buckling mechanism is anchored in the lumen.

In some embodiments, the anti-buckling member provides increased stiffness along the length of the sleeve. For example, the anti-buckling member can be a reinforcing member that is coupled to the sleeve. Alternatively, the anti-buckling mechanism can be a different material stiffer than the material of the sleeve.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is a cross-sectional diagram of an implantable anchored sleeve;

FIG. 1B is a cross-sectional diagram of the implantable anchored sleeve of FIG. 1A in a concentrically-everted state;

FIG. 1C is a cross-sectional diagram of the implantable anchored sleeve of FIG. 1A in a eccentrically-everted state;

FIGS. 2A and 2B are schematic diagrams of an implantable anchored sleeve according to one embodiment of the invention;

FIG. 3A is schematic diagram of an eversion pressure measurement test setup;

FIG. 3B is schematic diagram of an alternative eversion pressure measurement test setup;

FIGS. 4A and 4B are schematic diagrams of an exemplary implantable anchored sleeve according to one embodiment of the invention having a tapered section;

FIGS. 5A and 5B are schematic diagrams of an exemplary implantable anchored sleeve according to one embodiment of the invention having an eccentric-eversion resistant feature;

FIGS. 6A and 6B are schematic diagrams of an exemplary implantable anchored sleeve according to one embodiment of the invention having a tapered section and an eccentric-eversion resistant feature;

FIGS. 7A and 7B are schematic diagrams of an eccentric eversion measurement test setup;

FIG. 8 is schematic diagram of an exemplary embodiment of an implantable anchored sleeve including an eccentric eversion resistant feature and a wave anchor;

FIG. 9 is cross-sectional schematic diagram of a portion of the gastrointestinal tract illustrating the location of the exemplary implantable sleeve of FIG. 8; and

FIG. 10 is cross-sectional schematic diagram of an exemplary embodiment of an implantable anchored sleeve including an eccentric eversion resistant feature; and

FIG. 11 illustrates an exemplary embodiment of an anchored sleeve including an anti-buckling mechanism.

FIG. 12 illustrates an exemplary embodiment of an implantable anchored sleeve with a reduced diameter zone.

### DETAILED DESCRIPTION OF THE INVENTION

A description of preferred embodiments of the invention follows.

This invention relates to a method and device for implanting a sleeve within a natural body lumen of an animal, the sleeve including an anti-buckling mechanism to inhibit buckling and therefore eversion of the sleeve when implanted. In particular, the invention relates to a bypass sleeve adapted for use within the digestive tract of an animal. Some examples of such intestinal implants are described in U.S. patent application Ser. No. 11/000,099, filed Nov. 30, 2004, and entitled “Bariatric Sleeve”; U.S. patent application Ser. No. 11/001,794, filed Nov. 30, 2004, and entitled “Methods of Treatment Using a Bariatric Sleeve”; U.S. patent application Ser. No. 10/726,011, filed Dec. 2, 2003, and entitled “Anti-Obesity Devices”; U.S. patent application Ser. No. 10/810,317, filed Mar. 26, 2004, and entitled “Enzyme Sleeve”; and U.S. patent application Ser. No. 10/811,293, filed Mar. 26, 2004, and entitled “Anti-Obesity Devices” all incorporated herein by reference in their entirety.

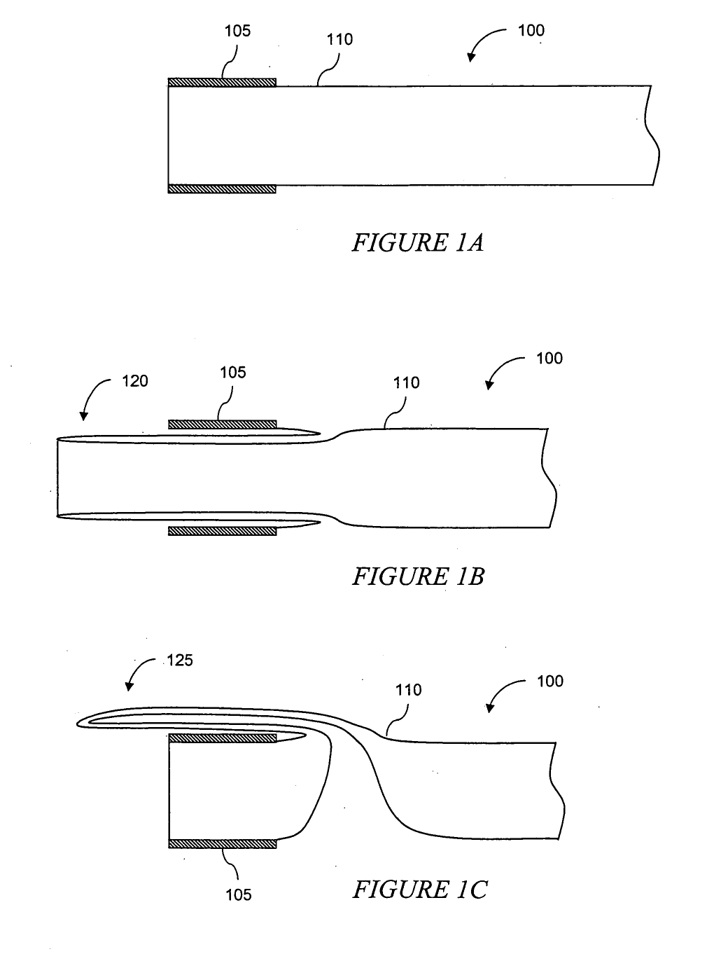

As illustrated in FIG. 1A, an exemplary gastrointestinal sleeve 100 includes a sleeve anchor 105 coupled to the proximal end of an elongated, thin-walled, flexible, floppy sleeve 110. The anchor can be, for example, a waveguide anchor as disclosed in U.S. application Ser. No. 10/858,852 filed on Jun. 1, 2004 claiming the benefit of U.S. Provisional Application 60/544,527 filed on Feb. 13, 2004, herein incorporated by reference in their entirety, or a stent. The sleeve is hollow with openings at both ends defining an interior lumen.

In this application, the sleeve is implanted within the intestine, such that chyme flowing within the intestine travels through the interior of the sleeve effectively bypassing that portion of the intestine. Preferably, the sleeve is thin-walled to so as to avoid irritating the intestine. Additionally, the thin-walled sleeve offers little resistance to peristaltic forces. Exemplary wall thicknesses are between 0.0003 and 0.0020 inches (i.e., 0.0076 and 0.051 mm).

Additionally, the sleeve material along the interior surface of the sleeve is smooth and slippery to avoid impeding the flow of chyme within the sleeve. Similarly, the exterior of the sleeve may also be smooth and slippery to promote the flow of material, such as digestive enzymes, between the exterior of the sleeve and the intestine wall. In some embodiments, the coefficient of friction of the sleeve material is about 0.2 or less. A suitable and preferred material for a sleeve is formed from an ePTFE/FEP bi-laminate material available from W. L. Gore & Associates Medical Products Division, Flagstaff, Ariz.

The sleeve anchor is adapted for removable attachment thereby securing at least a proximal portion of the sleeve to the intestine. Although the sleeve may be attached anywhere within the intestine, it is preferably implanted in the small intestine, distal to the pyloric sphincter between the pylorus and the ampulla of vater, with the attached sleeve extending distally into the intestine for a predetermined length. An example of such a device is described in U.S. patent application Ser. No. 10/858,851, filed on Jun. 1, 2004 and entitled “Intestinal Sleeve,” incorporated herein by reference in its entirety.

Although peristalsis provides a net resulting force directed antegrade from the stomach, there are times in the digestion cycle during which negative pressures or reverse peristalsis may occur. These negative or retrograde pressures may be the result of natural mixing waves, or other processes such as vomiting. The level of such pressures generated within the intestine are not well documented in the literature. Normal peristaltic pressures have been found to spike to 1.5-2.0 pounds-per-square-inch gauge (PSIG) (i.e., about 41.5-55.4 inches H₂O). It is expected that reverse peristalsis could produce similar spikes in pressure. If the pylorus is open, even slightly during this rise in pressure, there exists a driving force to push a gastrointestinal liner (i.e., sleeve) retrograde towards the stomach. Experiments in a porcine model have resulted in occasional vomiting that resulted in sleeve devices anchored in the duodenum to evert both through and around the anchor into the stomach. Once everted, the sleeve no longer functions and becomes obstructed.

The desirable features of being extremely thin-walled, flexible, floppy, and having a low friction coefficient all tend to make an intestinal sleeve more prone to buckling and therefore eversion. The sleeve often times has a tendency to “buckle” or bend within the intestine. This can interfere with the function of the sleeve. In order to prevent buckling of the sleeve, an anti-buckling mechanism can be provided. The anti-buckling mechanism tends to increase the stiffness of the sleeve, thereby preventing buckling.

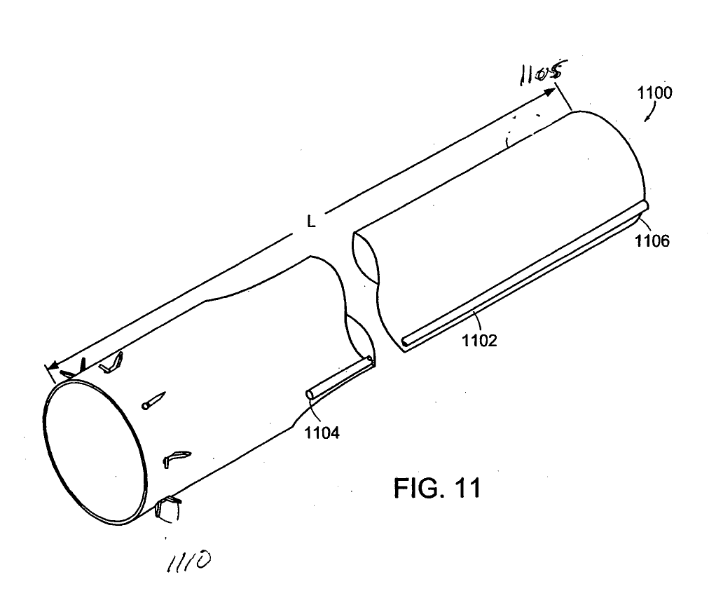

FIG. 11 illustrates a gastrointestinal device 1100 including an anti-buckling mechanism 1102. A flexible, anti-rotation, anti-buckling mechanism 1102 is attached to a sleeve 1105 and extends from below the distal end of an anchor having barbs 1110 along the length of the sleeve to the distal end of the sleeve 1105. In the embodiment shown, the anti-buckling mechanism 1102 is a guidewire device attached to the exterior surface of the outer layer of the flexible sleeve. Guidewire devices are well known to those skilled in the art. A first proximal end of the guidewire device 1104 is attached below the anchor and a second distal end of the guidewire device 1106 is attached to the distal end of the flexible sleeve 1105. The diameter of the guidewire ranges from about 0.010″ to about 0.016″.

The guidewire 1102 acts as the anti-buckling mechanism, by increasing the stiffness of the sleeve. The anti-buckling mechanism also, therefore, acts to inhibit eversion of the sleeve due to the increased stiffness.

At least two different eversion modes have been observed. A first eversion mode illustrated in FIG. 1B is referred to as a concentric eversion and is characterized by at least a portion of the sleeve 110 passing proximally through the center of the sleeve anchor 105. A second eversion mode illustrated in FIG. 1C is referred to as eccentric eversion and is characterized by at least a portion of the sleeve 110 passing proximally between the exterior surface of the anchor 105 and the interior surface (e.g., the tissue) of the body lumen within which the device 100 is implanted.

Eversions of a sleeve are more prone to occur when there is a relatively stiff section (e.g., the anchor) followed by a flexible section. The stiff section serves as a bending point or pivot for the flexible material resulting in a natural stress concentration. The stiff section must remain open during application of the pressure so the flexible material has an opening through which to evert. The present invention prevents such undesirable occurrences by providing a design feature that inhibits the sleeves from everting.

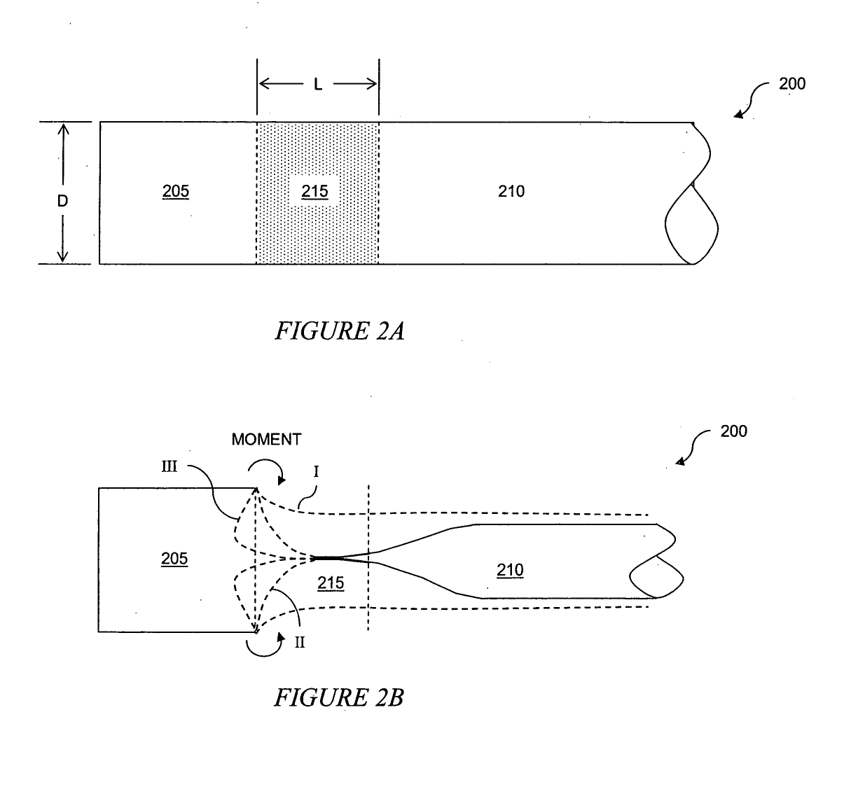

Eversion resistance can be accomplished by providing an anti-buckling mechanism which acts as an “eversion resistant” feature that inhibits eversion by increasing the stiffness of the sleeve. In the example shown in FIG. 2A, the eversion-resistant feature may be provided at least at the transition between the anchor and the free sleeve as illustrated in FIG. 2A. As shown, a gastrointestinal implant 200 includes a sleeve anchor 205 at its proximal end followed by an elongated sleeve 210 at its distal end. An eversion-resistant feature 215 is provided at the transition between the anchor 205 and the sleeve 210.

As retrograde force and/or pressure increases, the walls of the eversion-resistant feature 215 may experience a moment of force about a pivot formed at the intersection of the relatively stiff anchor 205 and the more flexible eversion-resistant feature 215 (i.e., there is a tendency for the device to fold in upon itself as shown in FIG. 1B). Depending upon the magnitude of the force, the moment may tend to cause at least a partial rotation of the wall of the eversion-resistant feature 215. However, because the eversion-resistant feature 215 is adapted to resist eversion, rotation may be limited to substantially less than 90°. This initial bending phase is referred to herein as a pre-eversion phase and is schematically illustrated as phase I in FIG. 2B.

As the retrograde force and/or pressure increases, bending of the eversion-resistant feature may continue, approaching 90°, until at least some of the interior surfaces of the eversion-resistant feature come into contact with each other. When the interior of the eversion-resistant feature 215 collapses upon itself, it is referred to as a collapsed phase and is schematically illustrated as phase II. It is believed that the resulting structure formed by the at least partially collapsed sleeve provides enhanced eversion-resistance performance. Namely, a collapsed portion of the device gains additional reinforcement from the collapsed region due at least partially to rotated material from one side of the device pushing against similarly rotated material from another side. Thus, further rotation about the pivot of either side is at least partially inhibited by the opposite sides pushing against each other. A similar process is relied upon in reed-type valves sometimes referred to as “duckbill valves.” Additionally, to the extent the surface material provides any non-insubstantial frictional coefficient, the resulting frictional force caused by overlapping layers of the material will resist movement of the material against itself and/or its surroundings, thereby inhibiting further eversion.

With an even greater retrograde force and/or pressure, bending of the eversion-resistant feature about the pivot may continue beyond 90°. As shown, the collapsed eversion-resistant feature 215 may begin to advance proximally into the interior aperture of the anchor 205. When a non-insignificant portion of the eversion-resistant feature 215 begins to advance proximally into the interior of the anchor 205, it is referred to as a partial-eversion phase and schematically illustrated as phase III. It is believed that the eversion-resistance performance remains enhanced during this phase as at least a portion of the device remains collapsed upon itself. Thus the reinforcing and/or frictional forces described above remain active. Consequently, there remains only a limited length of the device between the region of the collapse 215 and the pivot point, which limits partial eversion according to the length of this region. Of course, at sufficient forces and/or pressures, even the eversion-resistant feature will evert.

The eversion performance of a material can be characterized by its eversion pressure, which is the pressure required to evert a tube formed from the raw material. The eversion pressure is a measure of several properties of the material being affected at least by the material's stiffness and friction coefficient. Namely, raising either or both of a material's stiffness and friction coefficient yields higher material eversion pressures. Material stiffness is a function of at least the flexural modulus or hardness of the material and its wall thickness. The friction coefficient is also relevant because as the eversion starts, the material tends to roll at least partially upon itself. Once the material overlaps in this manner, any further movement requires that the material slide against itself. Thus, higher friction coefficient materials tend to increase frictional forces encountered by an everted sleeve, requiring increased forces to evert the materials once they have folded upon themselves.

The anti-buckling mechanism acting as an eversion-resistant feature may include one or more of the following attributes: increased stiffness or column strength, and an increased friction coefficient. An increased column strength resists that portion of the device 200 folding upon itself. Preferably, the length of this region ‘L’ is selected to allow at least a portion of the material to collapse fully on itself when a backpressure is applied. It is believed that such a collapse of the material forms a valve that can resist the pressure when the material is sufficiently stiff. The stiffness of the material is selected to promote its collapse and the formation of a valve at pressures at or near the eversion pressure of the otherwise unmodified raw sleeve material. To enable collapse upon itself, the length of the eversion-resistant feature 215 is greater than half the diameter of the internal lumen of the anchor 205 (i.e., L>D/2). Ideally, the eversion-resistant feature 215 also promotes collapse of the sleeve towards the elongated sleeve's central axis to prevent eccentric eversions.

One means of increasing the stiffness along the length of the eversion-resistant section 215 is to increase the material thickness. Increasing the thickness can be accomplished by layering the sleeve material upon itself until the desired thickness is attained. In some embodiments, the sleeve-anchoring device is encapsulated within two layers of sleeve material. Simply extending the region of the overlap a predetermined distance beyond the anchor itself provides a nice means of combining such functions. Alternatively, the eversion-resistant feature 215 can be formed using a second material having a higher modulus, thereby creating a relatively stiffer section.

Yet another means of increasing the material stiffness is providing reinforcing members coupled to the eversion-resistant section. For example, stiffness is increased by coupling one or more soft guidewires to the sleeve 210. At least one way to couple reinforcing members is to encase them within inner and outer layers of the sleeve material. Such an approach reduces the possibility that the reinforcing member will entrap chyme, impede peristalsis, and irritate the surrounding tissue.

The guidewire provides linear stiffness thereby acting as an ant-buckling mechanism and anti-eversion feature, while still allowing the section 215 to collapse and also providing little resistance to peristalsis. The guidewire is preferably oriented parallel to the central axis of the sleeve. The wire could be a vascular type guidewire commonly used to deliver catheters. These are typically constructed from stainless steel coils and having diameters between about 0.010 and 0.016 (i.e., 0.25 and 0.41 mm).

Materials such as soft, sticky silicone or polyurethane may be used in the anti-eversion feature 215. In some embodiments, one or more less-slippery materials are provided as a coating to the sleeve material. Alternatively or in addition, the friction coefficient of the eversion-resistant feature is increased by including a textured surface. Similarly, as the textured material collapses upon itself and attempts to roll inside out, the textured surface rubs against an adjacent surface to resist further sliding of the materials.

An exemplary embodiment of an implant device includes a sleeve formed from an ePTFE/FEP bi-laminate material available from W. L. Gore & Associates Medical Products Division, Flagstaff, Ariz. The sleeve is formed having an internal diameter of about 1 inch (i.e., about 25 mm) with an unmodified eversion pressure of about 3-7 inches H₂O. For the purposes of the testing, the length L of the eversion-resistant feature of the device was about 1.25 inches (i.e., about 3.2 cm) long. Additionally, the eversion-resistant feature was linearly tapered along its length from about 50 mm to about 25 mm in diameter. The number of layers of material used was varied from 2 covering the anchor, to 2 at the transition from the anchor to the tube, to 3 in the tube section. Each layer of material was about 0.0004 inches (i.e., about 0.0102 mm) thick. This construction resulted in an eversion pressure of the strain relief section of at least 30 inches H₂O but preferably 40-60 inches H₂O. Preferably, transition from the anchor to the sleeve is accomplished in a gradual manner. For example, the transition includes staggering the thickness changes.

In some embodiments, the thickness of the eversion-resistant section is 0.002-0.004 inches (i.e., about 0.051 to 0.102 mm) and requires about 4-8 layers of the base material. This construction results in an eversion pressure of the strain relief section of at least 30 inches H₂O. Devices have been made with pressures of 60 inches H₂O. The target specification is preferably between about 35-55 inches H₂O.

Animal testing in a porcine model has demonstrated that using a device having a concentric eversion pressure of 30-60 inches H₂O, eliminated the occurrence of concentric eversions. However, a new failure mode was observed during testing, which is referred to as eccentric eversion. Several attributes of the test devices appeared to contribute to the eccentric eversions.

The transition region became substantially stiffer as more layers of material were applied. Also, the surface area of the anchor increased as the relaxed diameter increased from 50 mm to 60 mm. This increases the effective force acting on the anchor legs due to the pressure within the duodenum. With sufficient forces, one or more of the anchor legs can be pushed away from the wall of the duodenum. With the anchor deformed in this manner, the relatively stiff reinforced sleeve section may bend in the direction of the pressure towards the opening formed by the moved anchor leg. Thus, the net result of increasing the stiffness of the transition region too much for a given stiffness of the anchor can lead to an increased susceptibility to eccentric eversions.

Susceptibility to eccentric eversion can be improved by decreasing the relative stiffness of the transition region while maintaining the increased relative stiffness of the proximal sleeve. For example, stiffness of the transition was decreased by providing only 2 layers of the sleeve material; whereas, the relative stiffness of the first 1-2 inches of the 25 mm tube was increased by adding 3 layers of the same material in that region. Beneficially, the resulting eversion pressure remains between about 30 and 60 inches H₂O while the likelihood of eccentric eversions is substantially reduced. Also, the softer transition region promotes collapse of the region concentrically, thereby preventing it from falling towards a side potentially leading to an eccentric eversion.

Thus, an anti-buckling mechanism and therefore anti-eversion feature is provided by a compound element consisting of at least two sections. The first can be a tapered section that transitions from the 50 mm anchor to the 25 mm sleeve. This section serves several purposes. First, it makes the transition in diameters. Additionally, it serves as a so-called low-pressure “crumple zone.” In other words, it collapses concentrically at low pressure without pulling the anchor away from the tissue surfaces. Preferably, the length of the crumple zone is no longer than the length of the anchor to avoid the crumple zone everting through the anchor. In some embodiments, the length of the crumple zone is about half the diameter of the sleeve. Then the second section is the stiffened sleeve section, which is drawn towards the center of the lumen by the collapse of the crumple zone. This area is stiff and therefore resists concentric eversion and buckling due to the increased stiffness. This section may be tapered from 3 layers to 1.

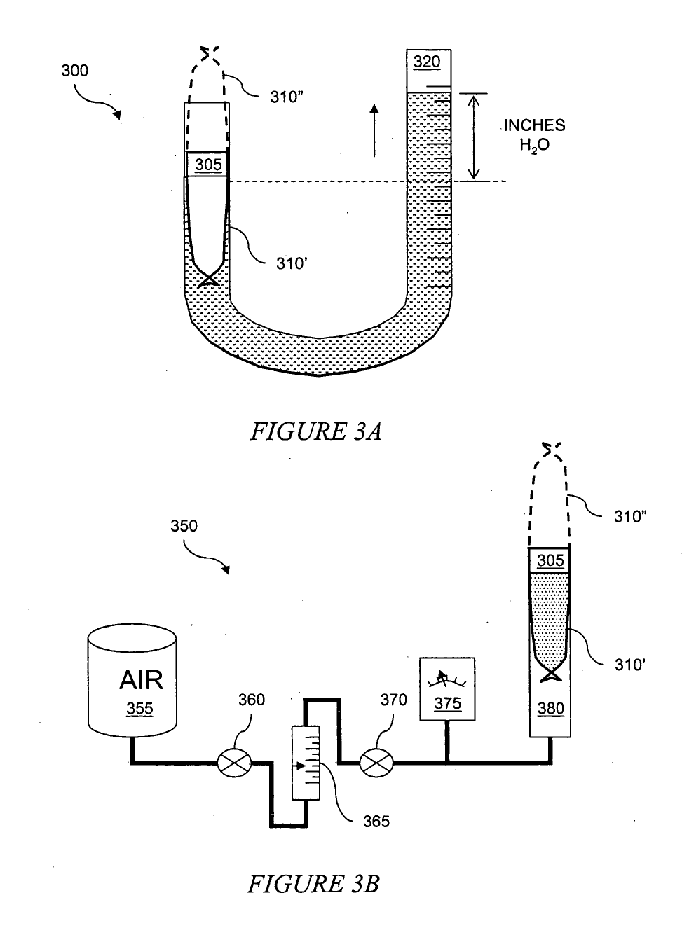

Measurement of concentric eversion-threshold pressure can be performed using a water-based test configuration measuring directly the inches of H₂O required to evert the device. As shown in FIG. 3A, the anchor 305 of a 25 mm diameter device is sealably attached to the interior of a 25 mm diameter silicone tube 320. The attached sleeve 310′ is tied off at some distance from the anchor 305 (e.g., about 6 inches from the anchor). The closed sleeve is extended within the tube 320 distal to the anchor 305. The tube 320 is bent into a ‘U’ shape with the device being placed in one of the vertical legs with other vertical leg being left open.

In operation, the tube 320 is partially filled with water from its open end. The water in the tube 320 represents a column of water applied to the distal side of the anchor 305. The open end of the tube is then raised with respect to the device, such that the potential energy of the displaced water provides a retrograde pressure upon the sleeve 310′. At some height, the sleeve 310″ everts through the anchor 305 as shown in phantom. The corresponding height of the water at which the sleeve 310″ everted is recorded as the corresponding eversion pressure in inches H₂O.

Another method of measuring concentric eversion-threshold pressure uses air rather than water. Air is preferred as it does not contaminate the tested materials, such that they can then be later used for implant. This set up is used to test the eversion pressure of either the raw material or the finished device. Raw material may be tested as an incoming quality assurance inspection to ensure consistency of the material. The overall concept described below is similar to the water-based test configuration.

Referring to FIG. 3B, the anchor 305 of a 25 mm diameter device is sealably attached to the interior of a 25 mm diameter silicone tube 380. The attached sleeve 310′ is tied off at some distance from the anchor 305 (e.g., 6 inches from the anchor). The closed sleeve 310′ is then extended within the tube 380 distal to the anchor 305. Air is supplied to the bottom of the tube 380 from a regulated air supply 355, such as a regulated air compressor through a flow-control system. The output of the air supply 355 is coupled through a needle valve 360 to one end of a flow meter 365. The other end of the flow meter 365 is coupled to one end of a check valve 370. The other end of the check valve is coupled to one end of the tube 380. A pressure-measuring device, such as a manometer 375 is coupled between the check valve 370 and the tube 380 to measure the pressure applied to the tube.

In operation, the check valve 370 is closed while a device under test is inserted into the tube 380. The device under test may be either samples of raw sleeve material or finished implants including eversion-resistant features. The needle valve 360 may be set to a pre-established flow rate such that the pressure will rise within the tube at a desired rate (i.e., not too fast to allow an operator to record pressure readings from the manometer 375. The check valve 370 is opened applying air pressure to the tube 380. As the pressure increases above the eversion-threshold pressure, the sleeve 310″ will evert through the center of the anchor 305 as shown in phantom. The corresponding maximum pressure at which the sleeve everted is recorded as the corresponding eversion pressure.

Either test configuration may be used to measure corresponding eversion pressures of devices with or without eversion-resistant features. Thus, comparative results between the two measurements provides a performance measure of any improvement provided by the eversion-resistant feature.

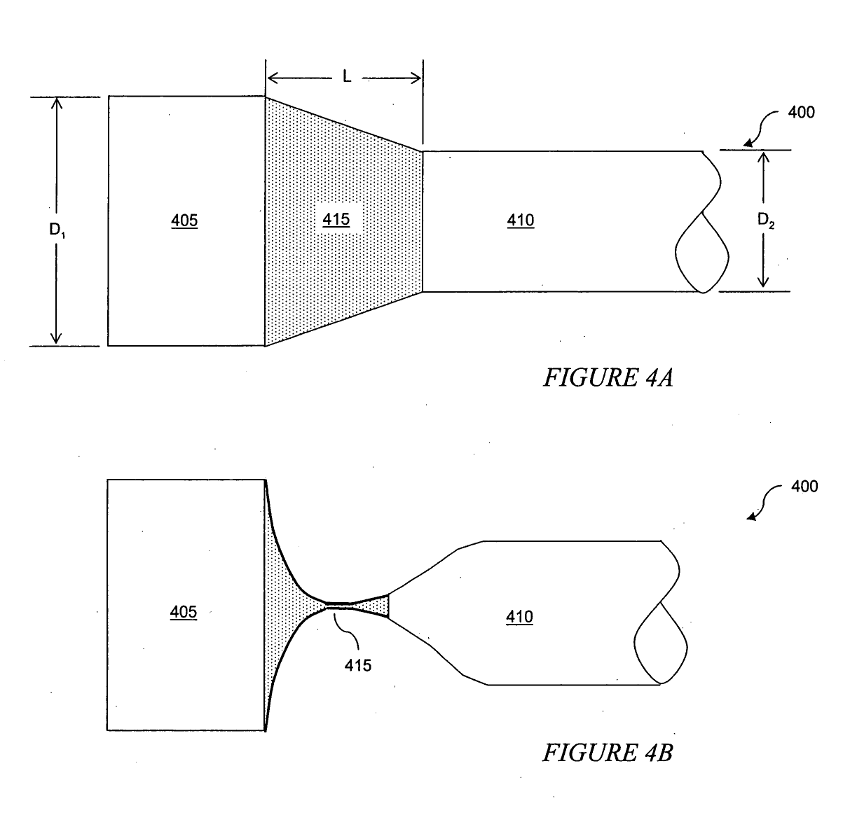

In some embodiments as shown in FIG. 4A, an implant device 400 includes an anchor 405 defining an interior lumen having a first diameter D₁ coupled to a sleeve 410 defines an interior lumen having a second diameter D₂. For example, the anchor includes a first diameter that is greater than the sleeve's diameter (i.e., D₁>D₂). This configuration is advantageous at least in gastrointestinal applications in which a seal between the anchor and the body lumen is desired. Thus, the anchor 405 functions in part as a radial spring, providing an outward force against the surrounding tissue when implanted. In order to provide the outward force, the resting diameter of the anchor is larger than the diameter when implanted.

An anti-buckling mechanism that is also a tapered eversion-resistant feature 415 can be applied between the anchor 405 and the sleeve 410, the feature 415 providing a transition from one diameter to another. For example, the eversion-resistant feature 415 is an open cone transitioning from D₁ to D₂. The eversion-resistant feature 415 can include any of the properties described above including increased stiffness and/or friction coefficient thus preventing buckling and therefore eversion. Similarly, these properties can be applied using any of the techniques described herein, the main difference being the tapered shape of the resulting treated area.

FIG. 4B illustrates deformation of the eversion-resistant feature 415 when subjected to retrograde pressures. Preferably, the eversion-resistant feature 415 collapses upon itself whereby the material properties resist eversion thereby blocking any opening through which the distal sleeve 410 may evert.

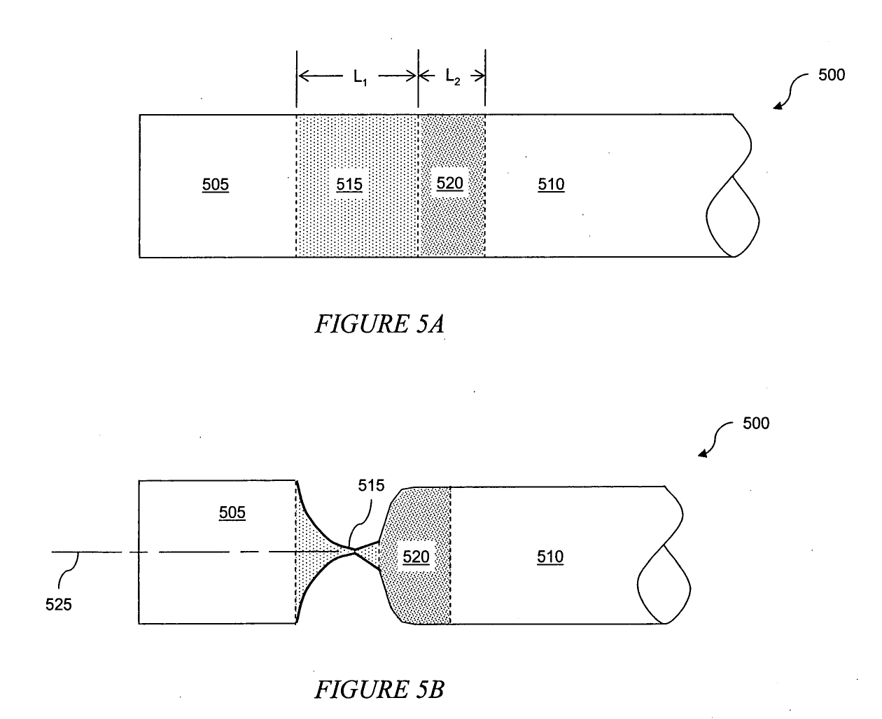

In some embodiments, an anti-buckling mechanism acting as an eversion-resistant feature is provided as a compound element providing different properties along different portions of the treated surface area. As shown in FIGS. 5A and 5B, an implant device 500 includes a proximal anchor 505 and a distal sleeve 510. The eversion-resistant feature provided between the anchor 505 and the sleeve 510 is applied resulting in at least two distinguishable regions. A proximal region 515 extends distally for a first length L₁ from the distal end of the anchor 505. A distal region 520 extends distally from the first region 515 for a second length L₂. The raw sleeve material extends distally from the distal end of the second region.

Such a compound eversion-resistant feature can provide eversion-resistance to both concentric eversions and to eccentric eversions. For example, the proximal region 515 can be configured as a so-called “crumple zone.” As the name suggests, when subjected to sufficient retrograde pressures, the proximal region 515 collapses upon itself as described above in reference to FIGS. 2 and 4. The distal region 520 can be configured as a so-called reinforced region having a higher eversion-resistance than the proximal region 515 to resist crumpling at the same pressure. The initial collapse of the proximal region 515 tends to center the distal region 520, such that further collapse of that region occurs towards the center rather than along the edge as the retrograde pressure continues to increase. Collapse of the distal region 520 ultimately blocks the central lumen without everting fully, thereby prohibiting further eversion of the sleeve 510 through the blocked lumen.

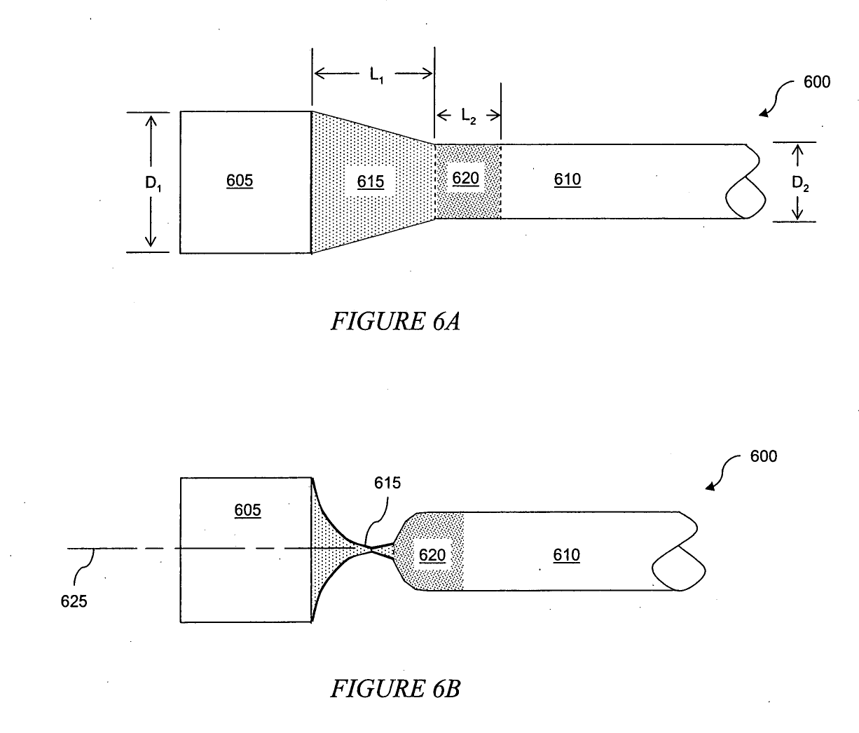

A tapered device having a compound eversion-resistant feature is illustrated in FIGS. 6A and 6B. The device 600 includes a proximal anchor having a first diameter D₁ (e.g., about 50 mm) coupled through an eversion-resistant feature to the proximal end of an elongated sleeve having a second diameter D₂ (e.g., about 25 mm). Typically, the sleeve's diameter is less than that of the anchor 605 (i.e., D₁>D₂). The compound eversion-resistant feature includes a proximal region 615 extending for a first length L₁ (e.g., about 1.5 inches) followed by a distal region 620 extending for a second length L₂ (e.g., about 1.0 inch).

The proximal region 615 can be configured as a crumple zone and the distal region 620 can be configured as a reinforced region. In the presence of sufficient retrograde pressures, the proximal region 615 collapses upon itself first while the distal region remains substantially open. As the pressure continues to increase, the distal region 620 also collapses upon itself, being substantially centered by the initially-collapsed crumple zone 615, thereby avoiding an eccentric eversion.

In some embodiments, tapering from the first D₁ to D₂ is accomplished in the proximal region 615. It is believed that applying a taper to this region may further enhance performance of the eversion-resistant feature by focusing collapse of the material towards the device's longitudinal axis.

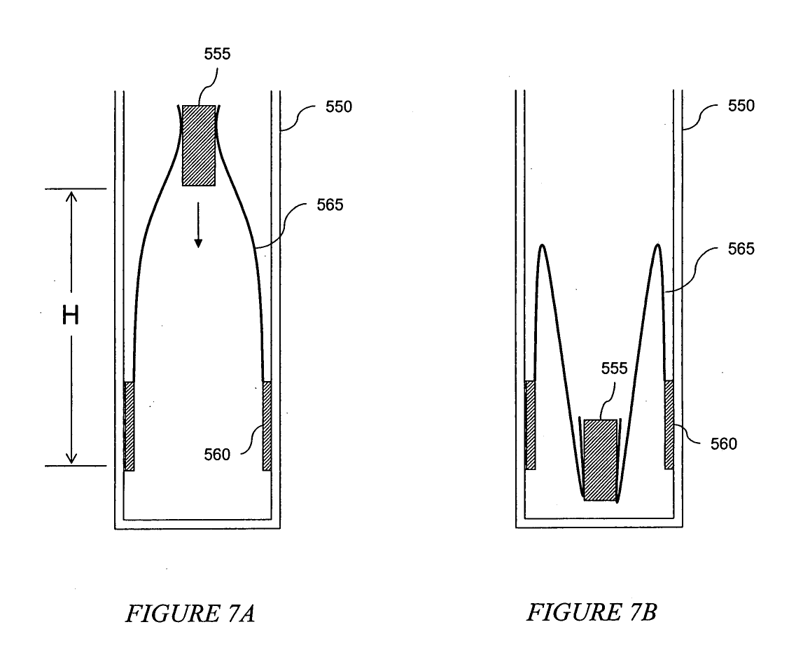

Measurement of eccentric eversion susceptibility can be accomplished using an eccentric-measurement test setup. An exemplary test setup is illustrated in FIGS. 7A and 7B. The anchor of an implant device under test is coupled to the interior of a large-diameter silicon tube (e.g., about 40 mm for a 50 mm diameter anchor). A weight is then attached to a distal end of the sleeve at a predetermined distance from the anchor. The weight is raised above the anchor to fully extend the sleeve. For example, the weight can be a metal rod that is placed inside the sleeve, coupled to the sleeve, and dropped from a height of about 6 inches (i.e., about 15 cm) towards the anchor. The metal rod is relatively narrow. For example, a metal rod about 0.5 inches (i.e., about 13 mm) in diameter that weighs about 0.6 pounds (i.e., 0.23 kg) was used for test results provided herein.

The weight is dropped towards the anchor and depending upon the device under test, the weight may travel through the center of the anchor resulting in a concentric eversion, or the weight may travel towards a side of the anchor resulting in an eccentric eversion. The test is repeated a predetermined number of times for the same device under test. Eccentric eversion susceptibility is determined as the percentage of total tries resulting in an eccentric eversion.

Thus, this test can be used to measure the eccentric eversion susceptibility of different devices and is useful in identifying features that reduce or eliminate the 5 eccentric eversion failure mode. Four different devices were tested using the test configuration of FIGS. 7A and 7B. The devices are described in Table 1.

TABLE 1
Devices Under Test

| Design | | | Material | Layering |
|---|---|---|---|---|
| # | Anchor design | Eversion design | thickness | method |
| 1 | 60 mm OD × | Single cone | 0.0010″- | Wrapped |
| | 0.020″ wire | transition element | 0.0015″ | |
| | diameter | and short cylinder | | |
| 2 | 50 mm OD × | Single cone | 0.0015″- | Template |
| | 0.023″ wire | transition element | 0.0020″ | |
| | diameter | | | |
| 3 | 50 mm OD × | Single cone | 0.0010″- | Wrapped |
| | 0.023″ wire | transition element | 0.0015″ | |
| | diameter | and short cylinder | | |
| 4 | 50 mm OD × | Single cone | Cone is | Template |
| | 0.023″ wire | transition element | 2 layers | |
| | diameter (most | and long cylinder | (0.0010″) | |
| | recent design) | | Cylinder is | |
| | | | 3 layers | |
| | | | (0.0015″) | |

Exemplary data resulting from 30 attempts per device for each of the 4 different devices is summarized in Table 2.

TABLE 2
Eccentric Test Results

| Device | Concentric | Eccentric | % Eccentric |
|---|---|---|---|
| Design 1 | 20 | 10 | 33.3% |
| Design 2 | 13 | 2 | 13.3%* |
| Design 3 | 30 | 0 | 0% |
| Design 4 | 30 | 0 | 0% |

*15 tries only - device broke

These tests showed that the eversion-resistant features of devices 3 and 4 are much less susceptible to the eccentric-eversion failure mode. These data also are supported by animal evaluations. Designs 1 and 2 had high rates of eccentric eversion in pigs. Design 3 was an early design in which eversions were very rare. Design 4 has also resulted in a device in which eversions are rare in animal testing.

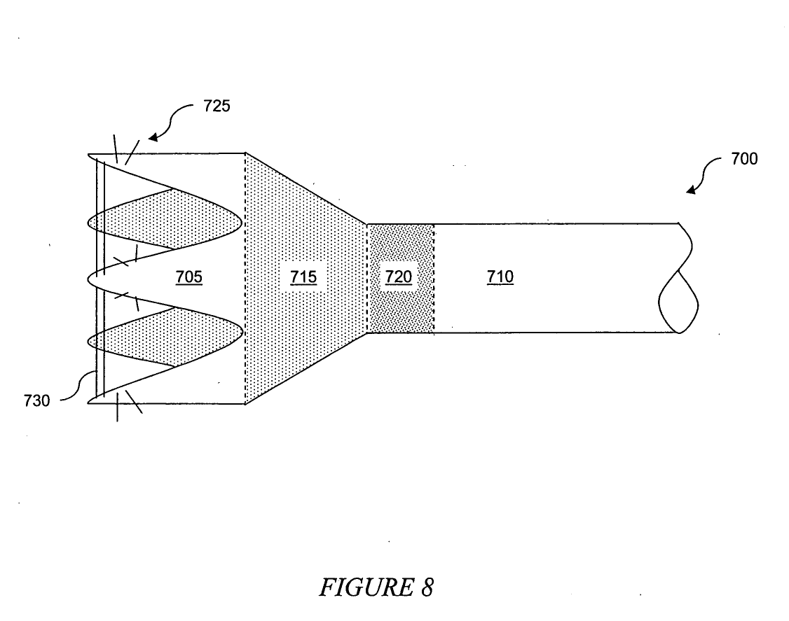

An embodiment combining a wave anchor with a compound eversion-resistant feature is illustrated in FIG. 8. The device is similar to that described above in reference to FIG. 6A in that it includes a proximal anchor 705 having a first diameter and a distal elongated sleeve 710 having a second diameter less than the first. A compound eversion-resistant feature includes a proximal region 715 adjacent to the anchor and tapered between the first and second diameters. A reinforced region 720 is provided between the proximal region 715 and the proximal sleeve 710. The anchor 705, however, is illustrated in more detail. In particular, the anchor can be a wave anchor defining multiple oscillations about a central lumen as described in U.S. application Ser. No. 10/858,852, filed on Jun. 1, 2004, and entitled “Method and Apparatus for Anchoring Within the Gastrointestinal Tract” incorporated herein by reference in its entirety. As shown, the proximal portion of the sleeve can be tailored to the boundary defined by the anchor resulting in the tulip-petal shape. The anchor, when implanted is reduced in diameter slightly by the local anatomy of the body lumen. Beneficially, the outward radial spring force provided by the partially-compressed anchor results in a sealable connection between the proximal end of the device and the interior surface of the body lumen.

The spring force of the anchor provides some anchoring force to maintain the anchor in a predetermined location. However, the anchor can be attached to the local anatomy using one or more external connecting means. For example, the anchor can be sutured in place, coupled using surgical staples, and/or coupled using surgical adhesives. Preferably, the anchor is attached to the anatomy in a removable fashion. For example, the anchor can optionally include one or more barbs 725 or spines protruding outward and adapted to engage the surrounding muscular tissue.

Alternatively or in addition, the device can include one or more features adapted to facilitate removal of the device. For example, the device can include one or more drawstrings 730 at its proximal end. The drawstrings are slideably attached to the proximal ends of the anchor and are adapted to centrally collapse the anchor when suitably engaged. Preferably, the collapse pulls any barbs out of the surrounding tissue prior to removal to avoid unnecessary tissue damage. A separate removal device can then be used to remove the device as described in pending U.S. Provisional Application No. 60/663,352, filed on Mar. 17, 2005, and entitled “Removal and Repositioning Devices,” incorporated herein by reference in its entirety.

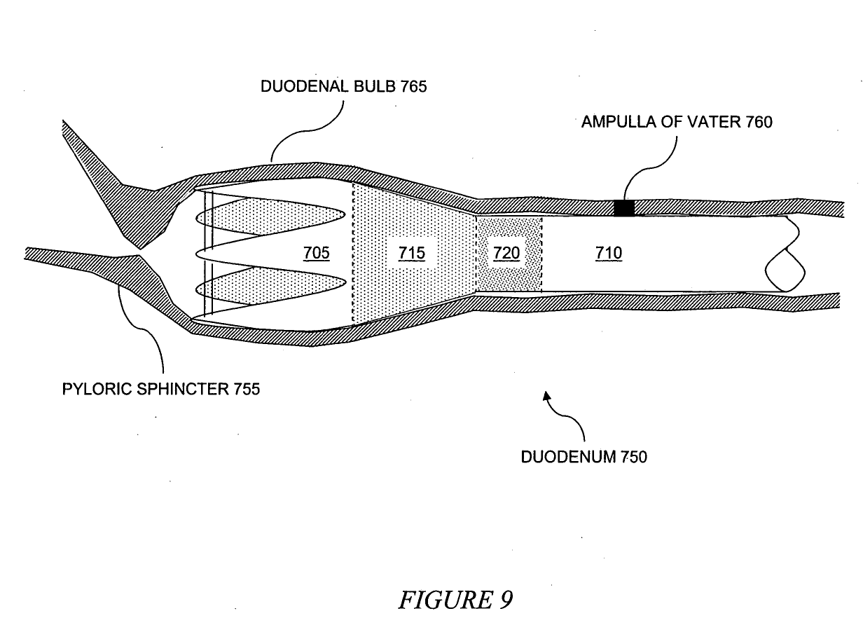

FIG. 9 shows a cross-sectional view of a portion of a duodenum 750 with a device implanted therein. The anchor 705 is situated in the proximal duodenum in an area referred to as the bulbous duodenum 765, located distal to the pyloric sphincter 755 and proximal to the ampulla of vater 760. The anchor 705 is partially compressed resulting in a fluid seal between it and the surrounding intestine wall. The sleeve 710 extends distally into the duodenum 750 and, depending upon its length, beyond the duodenum into distal parts of the small intestine not shown.

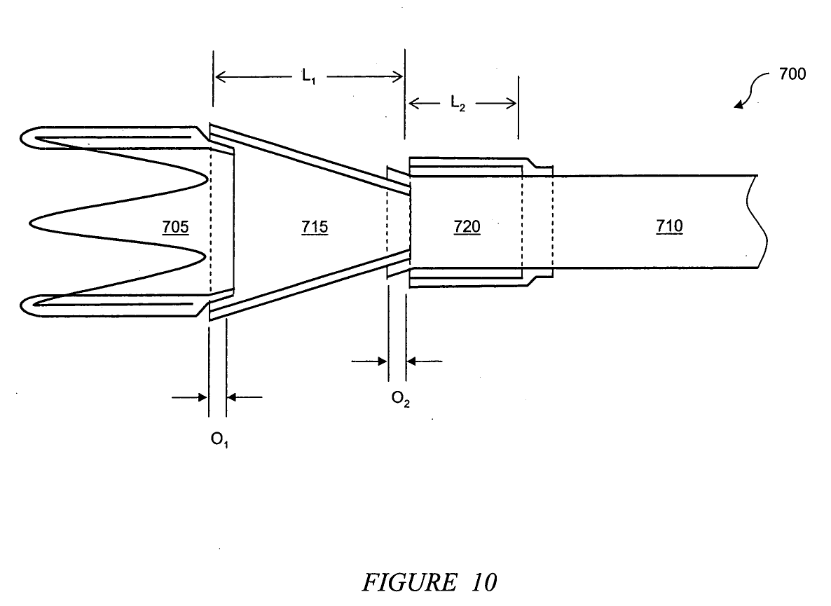

FIG. 10 shows a cross section of one embodiment of the sleeve 700 shown in FIG. 9 using overlapping material to form the different regions of the compound eversion feature. Starting at the proximal end, a wave anchor 705 is surrounded by an inner and outer layer of the sleeve material. The proximal anti-eversion region 715, or tapered crumple zone, is similarly formed using two layers of the same sleeve material. Preferably, some amount of overlap O₁ is provided to facilitate attachment of the covered anchor 705 to the proximal end of the crumple zone 715. For example, the two regions may be attached using an adhesive. Alternatively or in addition, the two regions may be attached using a mechanical fastener such as a suture. Preferably, however, thermal boding is used to sealably connect the two regions together along the periphery of the device within the overlapping region O₁.

A proximal end of the sleeve similarly overlaps a distal end of the crumple zone by a length O₂ to facilitate attachment of the two regions. Any of the above means of attaching can be used to form the attachment. A second and third layers are added just distal to the end of the crumple zone 715, thereby forming a reinforced region 720 having three layers of sleeve material. As shown, the outer-most layer 725 of the reinforcing region 720 may extend beyond the second layer 727 and attach to the outer surface of the sleeve 710 to form a smooth transition.

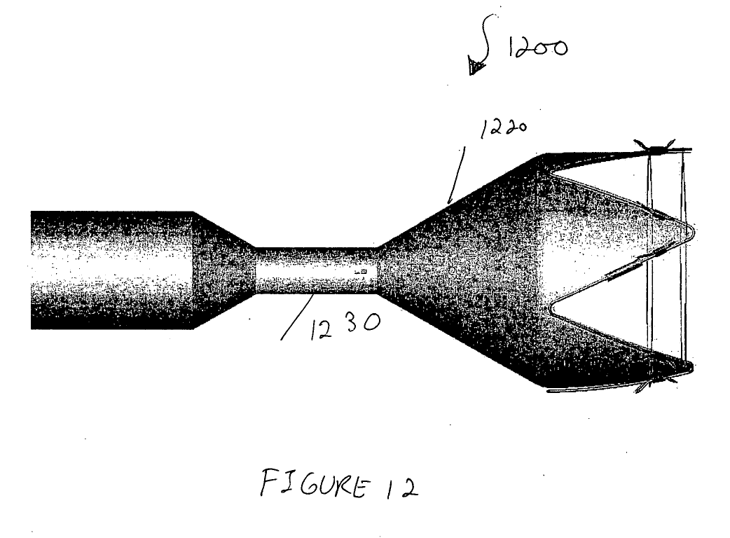

FIG. 12 shows an alternative embodiment that resists buckling and therefore eversion, of a gastrointestinal device 1200. A proximal portion of a sleeve 1220 can have a reduced section 1230 that is smaller than the diameter of the distal length of sleeve 1220. Both the diameter and length of the reduced section 1230 can be varied to achieve the desired eversion resistance pressure.

Because the diameter of the reduced section 1230 is significantly smaller than the diameter of the distal sleeve segment, it is stiffer and the pressure required to evert the distal sleeve segment through the reduced segment 1230 is significantly higher than it would be with no diameter reduction. Typically, the length of the reduced section 1230 is very short in comparison to the length of the distal end of the sleeve 1220. For example, in one embodiment the length and diameter of the distal end of the sleeve 1220 were respectively 22 inches and 1 inch, while the length and diameter of the reduced section were respectively 1 inch and 0.4 inches. These ratios were successfully tested and demonstrated in a pig model, and demonstrated an increased eversion resistance pressure of greater than 40 inches of water. Additionally, the smaller diameter and length of the reduced portion, along with resisting buckling and therefore eversion, serves as a flow restrictor that increases satiety as disclosed in U.S. application Ser. No. 10/811,293 filed on Mar. 26, 2004, claiming the benefit of U.S. Provisional Application No. 60/471,413, and as disclosed in U.S. application Ser. No. 11/330,705, filed on Jan. 11, 2006, claiming the benefit of U.S. Provisional Application No. 60/662,570, filed on Mar. 17, 2005 and U.S. Provisional Application No. 60/645,296, filed on Jan. 19, 2005, and incorporated herein by reference in their entirety.

Although a gastrointestinal sleeve is described as an exemplary embodiment, other applications include arterial grafts, esophageal prostheses, and other gastrointestinal prostheses, such as biliary sleeves.

While this invention has been particularly shown and described with references to preferred embodiments thereof, it will be understood by those skilled in the art that various changes in form and details may be made therein without departing from the scope of the invention encompassed by the appended claims.

What is claimed is:

1. A device for implanting within an animal body comprising:
an anchor adapted for attachment within a natural body lumen;
a thin-walled, floppy sleeve open at both ends and defining a lumen therebetween; and
an anti-buckling mechanism extending from below a distal end of the anchor along a length of the sleeve.

2. The device of claim 1, wherein the length of the sleeve extends to the distal end of the sleeve.

3. The device of claim 1, wherein the anti-buckling mechanism comprises increased stiffness relative to the sleeve's stiffness.

4. The device of claim 3, wherein the increased stiffness is provided by a different material than that of the sleeve.

5. The device of claim 4, wherein the anti-buckling mechanism is a wire.

6. The device of claim 3, wherein the increased stiffness is provided by a reinforcing member.

7. A method for inhibiting buckling of a sleeve device when implanted within a natural body lumen comprising:
providing an anti-buckling mechanism extending from below a distal end of an anchor along a length of a thin walled, floppy sleeve; and
anchoring a proximal end of the sleeve with the anti-buckling mechanism in the lumen.

8. The method of claim 7, further comprising the steps of increasing the stiffness of the thin-walled, floppy sleeve.

9. The method of claim 7, wherein the anti-buckling mechanism is a different material relatively stiffer than the thin-walled, floppy sleeve.

10. The method of claim 7, wherein the anti-buckling mechanism is a reinforcing member coupled to the thin-walled, floppy sleeve.

11. A device for implanting within an animal body comprising:
means for anchoring a proximal end of the device within a natural body lumen, the device including an elongated, thin-walled, flexible, floppy sleeve open at both ends and defining a lumen therebetween, a substantial length of the sleeve having material characteristics that result in the sleeve being prone to buckle; and
means, proximal to the anchoring means, for resisting buckling of the thin-walled sleeve.
